(19) United States
(12) Patent Application Publication
(10) Pub. No.: US 2016/0243376 A1
(43) Pub. Date: Aug. 25, 2016

(54) Magnetic Stimulator Arrangement

(71) Applicant: THE MAGSTIM COMPANY LIMITED, Whitland, Dyfed (GB)

(72) Inventor: Mark Phillips, Whitland (GB)

(21) Appl. No.: 15/026,211

(22) PCT Filed: Sep. 30, 2013
(86) PCT No.: PCT/GB2014/052938

§ 371 Date: Mar. 30, 2016

(30) Foreign Application Priority Data
Oct. 1, 2013 (GB) 1317367.9

(51) Int. Cl. A61N 2/02; A61N 2/00

(52) U.S. Cl. CPC A61N 2/02; A61N 2/006

(57) ABSTRACT

The present invention relates to a magnetic stimulator arrangement for the magnetic stimulation of neuromuscular tissue. Magnetic stimulators of this kind achieve stimulation by the creation of a rapidly changing magnetic field in the vicinity of the tissue. Electric currents thereby induced in the tissue cause stimulation thereof. The present invention comprises a magnetic stimulator apparatus for the magnetic stimulation of neuromuscular tissue comprising a flexible conductor arranged to define a multiple turn coil and a light guide configured to preferentially fail in the event of at least partial failure of the conductor.

The present invention relates to a magnetic stimulator arrangement for the magnetic stimulation of neuromuscular tissue.

Magnetic stimulators of this kind achieve stimulation by the creation of a rapidly changing magnetic field in the vicinity of the tissue. Electric currents thereby induced in the tissue cause stimulation thereof.

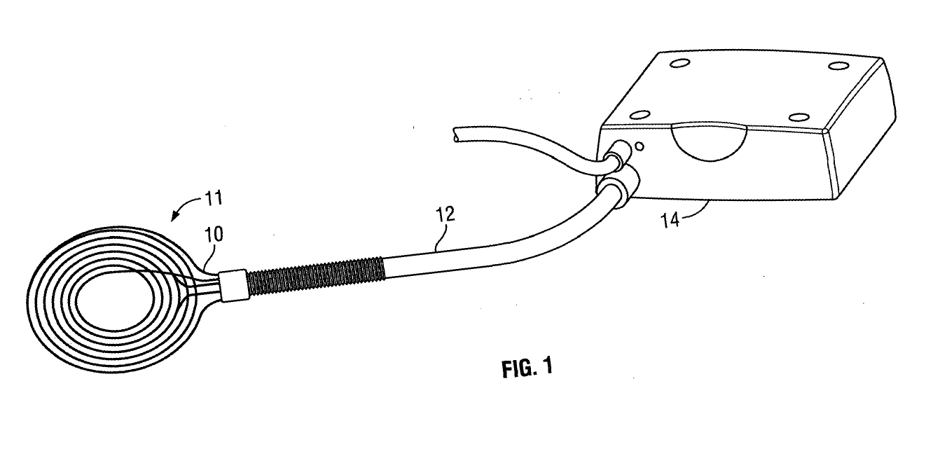

Known magnetic stimulator arrangements such as disclosed in GB2415632 comprise a charging circuit for a discharge capacitor, a discharge control such as a controlled rectifier for allowing discharge of the capacitor through the stimulating coil, and other circuit elements for limiting the effect of undesirable electrical transience. The coil may assume a variety of forms such as a self-supporting flexible conductor in a flexible insulating sheath which can be adjusted by hand to conform a coil to the contours of a selected part of the human body. Such an arrangement is presented in FIG. 1 which is a schematic representation of a prior art magnetic stimulator arrangement. There is a coil (11) and neck portion (10) which extends to a flexible insulating conduit (12). A housing (14) is provided which may accommodate a heat exchanger for cooling the coil (11), a pump and a reservoir, and the stimulator arrangement. The stimulator arrangement comprises a high voltage power supply which charges a reservoir capacitor. Charge is transferred from the reservoir capacitor via a transfer capacitor to a discharge capacitor which at the appropriate intervals is discharged into the simulating coil (11). A suitable circuit of one form of a magnetic stimulator suitable for use in the present invention is described, for example, in GB2415632 and GB2298370.

An alternative coil configuration is to provide side-by-side coils wherein the windings or turns of each individual coil may be in the same direction. The coils are substantially provided in the same plane and may be housed in a substantially rigid housing. This configuration of adjacent side-by-side coils with a single winding direction will be described in the exemplary embodiment of the present invention, however, it will be appreciated that the present invention may equally apply to the configuration as identified in the prior art in FIG. 1.

A significant problem associated with magnetic stimulator arrangements is the possibility of fracture of the elongate conductor that makes up the turns of the coil or coils. The turns of the coil undergo many cycles of heating and cooling and for certain applications also involve manipulation and adjustment to conform the coil to the contours of a selected part of the human body. Accordingly, the elongate conductor undergoes fatigue which eventually leads to failure. If failure occurs then the electric circuit is broken meaning that a fuse in the power source blows and due to the high voltage power supply, blowing of the fuse causes loud noise to be generated. This is undesirable if the apparatus is being used for a treatment on a patient. Further, fracture of the coil may lead to damage of the housing, potential for causing a patient discomfort, and interruption of a carefully planned treatment cycle.

The present invention overcomes these problems.

According to the present invention there is a magnetic stimulator apparatus for the magnetic stimulation of neuromuscular tissue comprising a flexible conductor arranged to define a multiple turn coil and a light guide configured to preferentially fail in the event of at least partial failure of the conductor.

Also according to the present invention there is a magnetic stimulator apparatus for the magnetic stimulation of neuromuscular tissue comprising a flexible conductor arranged to define a multiple turn coil and a light guide contiguous with at least a portion of the flexible conductor.

Accordingly it is beneficial that the light guide and flexible conductor are at least partially in communication and share a common border.

The light guide is therefore beneficially sacrificed.

There is preferably further provided an arrangement to provide an indication in the event of failure of the light guide. Such an indication may be, for example, an audible or visual warning to an operator of the apparatus. Operation of the apparatus can then be paused or shut off for maintenance to the flexible conductor to be carried out or alternatively a replacement coil can be used.

The light guide is beneficially in communication with and beneficially contiguous with at least a portion of the flexible conductor. Even more preferably the light guide is in direct physical communication with the flexible conductor. It is beneficial that there is direct physical communication as this will provide an immediate indication in the event of failure of the flexible conductor. For example, failure of the flexible conductor may commence with fraying due to fatigue. Minor fraying of the flexible conductor in communication with the light guide will cause fracture of the light guide. Fracture of the light guide causes the operator of the apparatus to be informed meaning that operation of the apparatus can be paused or terminated.

The light guide is beneficially arranged to fracture in the event of at least partial failure of the conductor.

The light guide is beneficially substantially aligned with at least a portion of the flexible conductor. The light guide may, therefore, effectively follow the contours of at least a portion of the flexible conductor.

The flexible conductor beneficially further comprises a neck portion. The multiple turn coil may effectively form a head portion. The neck portion beneficially comprises a longitudinal length and the light guide substantially aligns with at least a portion of the neck portion. The light guide beneficially aligns substantially parallel to the flexible conductor defining the multiple turn coil and beneficially aligns substantially parallel to at least a portion of the neck portion.

The light guide is beneficially an elongate flexible element.

It is beneficial that the light guide is at least partially secured and preferably adhered to the flexible conductor. A number of alternative adhesives may be utilised with an example being cyanoacrylate. This adhesive is beneficially provided in liquid form and may be used to adhere the light guide to at least a portion of the flexible conductor, preferably both in the multiple turn coil and the neck portion.

The light guide is preferably a light pipe and is preferably a fibre optic cable. Light is beneficially passed through the light guide and failure of the light guide results in an interruption causing prevention of light transmission. The apparatus beneficially comprises an arrangement for transmitting light through the light guide and receiving light from the light guide. In the event of failure of the light guide there is provided an indication of the failure. This may be achieved by a light sensor receiving light that has been transmitted along the light guide. In the event of a threshold light intensity not being achieved the light guide is deemed to have failed and an indication is given to an operator. At this time the power source is also beneficially switched off thus preventing use of the apparatus.

A flexible sheath is beneficially provided to house the flexible conductor and the light guide.

The apparatus preferably further comprises a charging circuit, a capacitor and a discharge control for allowing discharge of the capacitor through the flexible conductor.

There are preferably provided two adjacent multiple turn coils. The two coils are beneficially provided in a side-by-side configuration, preferably substantially in the same plane. The conductor preferably includes a bridge extending between the coils. The light guide is beneficially in communication with the bridge and even more beneficially aligns in communication with at least a portion of the bridge. This is beneficial as even though a first and second coil that are beneficially in a side-by-side configuration may be relatively secured to each other through being housed in a rigid housing, fatigue has particularly been found to be most significant at this bridge.

Also according to the present invention there is a method of identifying at least partial failure of a flexible conductor in a magnetic stimulator arrangement for the magnetic stimulation of neuromuscular tissue, the flexible conductor arrangement to define a multiple turn coil, the method comprising the step of monitoring for failure of a light guide wherein the light guide is arranged to preferentially fail in the event of at least partial failure of the flexible conductor.

The present invention will be described by way of example only with reference to the accompanying drawings in which:

FIG. 1 is a schematic prior art apparatus.

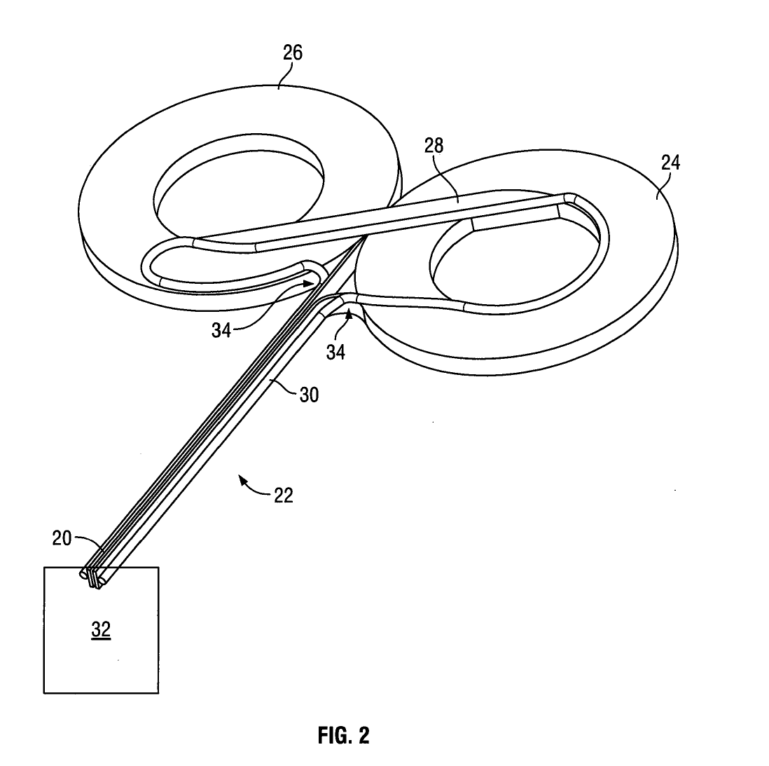

FIG. 2 is a schematic perspective view of an apparatus according to an exemplary embodiment of the present invention.

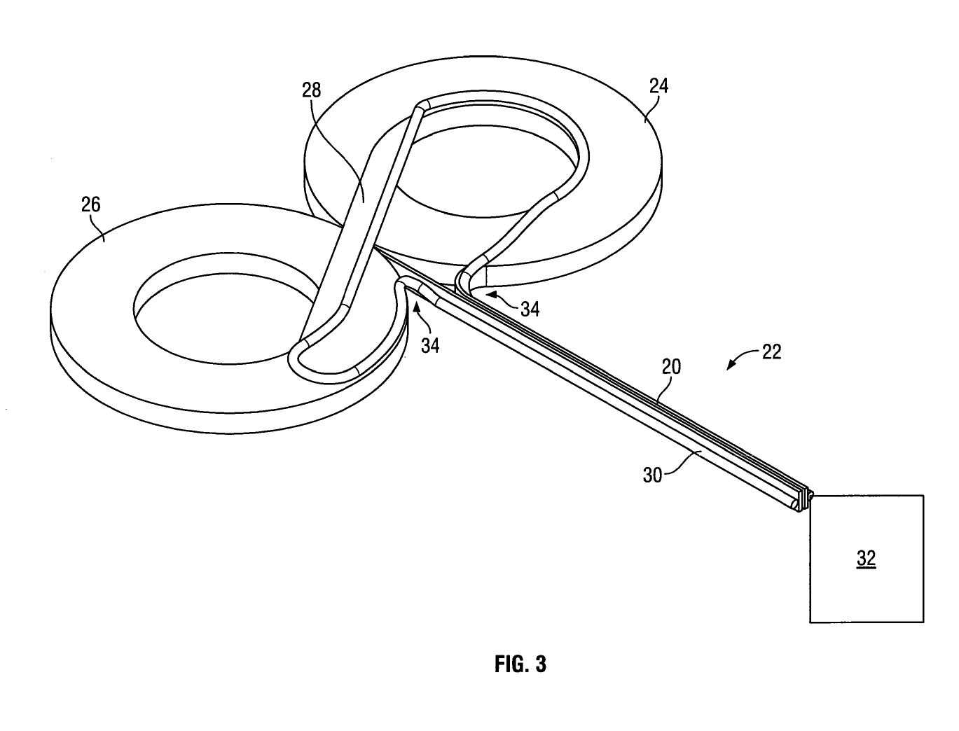

FIG. 3 is a second schematic view of an apparatus according to an exemplary embodiment of the present invention.

Referring to FIGS. 2 and 3 there is provided a flexible conductor (20) which is elongate and extends via a neck portion (22) to a first multiple turn coil (24), a second multiple turn coil (26) and back via the neck portion (22). The elongate conductor (20) may be formed of a conductor preferably comprising copper and in the exemplary embodiment shown is substantially rectangular in cross-section having a height of approximately 6 mm and a width of approximately 1.75 mm. The elongate conductor (20) extends anticlockwise around the first multiple turn coil (24) and passes to the second multiple turn coil (26) via a bridge (28). The elongate conductor (20) passes around the second multiple turn coil (26) in a clock wise direction and extends back to the neck portion (22) providing a return to the power supply (not shown). The apparatus represented in FIGS. 2 and 3 may be housed in a flexible sheath (not shown) allowing manipulation of the multiple turn coils (24,26) and neck portion (22), or alternatively may be housed in a substantially rigid housing. It will be appreciated that alternative coil turn configurations are possible such as turns in opposite directions to those described, or turns commencing radially outwardly to radially inwardly for example.

Due to use and the associated fatigue involved with multiple heating and cooling and in some embodiments conforming the apparatus to the contours of a selected part of the human body, the elongate conductor (20) is susceptible to failure. Accordingly, a light guide (30) is provided. The light guide (30) extends from a light source (32) including a light emitter and passes light through the light guide (30) in a loop and returns back to a light sensor that may be provided within the housing for the light source (32). In the event of failure of the light guide (30) through fracture light intensity transferred back to the sensor is reduced and if reduced below a threshold value an indication is provided of this failure. Such an indication may be an audible warning or a visual warning provided to the operator. As the light guide (30) extends around the likely points of failure of the elongate conductor (20) (particularly the bridge (28) and neck portion (22)) at least partial failure of the elongate conductor (20) at these weak points causes fracture of the light guide (30) leading to the operator being informed of the imminent failure. Operation of the apparatus may then automatically terminate through switching off the power for example for replacement or repair of the flexible conductor.

The light guide (30) is beneficially aligned with a part of the elongate conductor of the neck portion (22) and aligns with at least a portion of a turn of a coil in both the first multiple turn coil (24) and second multiple turn coil (26). The light guide (30) beneficially also extends and substantially aligns with at least a portion of the bridge (28). As the first and second multiple turn coils (24,26) may be manipulated relative to each other, the susceptibility to failure of the bridge (28) is further increased. Furthermore, the neck portion (22) is susceptible to failure in particular especially around the intersection (34) with the first and second multiple turn coils (24,26).

The light guide (30) is beneficially adhered or bonded directly to the elongate conductor (20). A number of suitable adhesives exist, an example being cyanoacrylate which is sold, for example, under the trade name ‘Loctite 406’.

The present invention has been described by way of example only and it will be appreciated by the skilled addressee that modifications and variations may be made without departing from the scope of protection afforded by the appended claims.

1. A magnetic stimulator apparatus for the magnetic stimulation of neuromuscular tissue comprising:
a flexible conductor arranged to define a multiple turn coil; and
a light guide.

2. The magnetic stimulator apparatus according to claim 1, wherein the light guide is contiguous with at least a portion of the flexible conductor.

3. The magnetic stimulator apparatus according to claim 25, comprising an arrangement to provide an indication in the event of failure of the light guide.

4. The magnetic stimulator apparatus according to claim 25, wherein the light guide is arranged to fracture in the event of at least partial failure of the conductor.

5. The magnetic stimulator apparatus according to claim 25, wherein the light guide is a sacrificial light guide.

6. The magnetic stimulator apparatus according to claim 24, wherein the light guide is in communication with at least a portion of the flexible conductor.

7. The magnetic stimulator apparatus according to claim 24, wherein the light guide is contiguous with at least a portion of the flexible conductor.

8. The magnetic stimulator apparatus according to claim 7, wherein the light guide is in direct physical communication with at least a portion of the flexible conductor.

9. The magnetic stimulator apparatus according to claim 25, wherein the light guide substantially aligns with at least a portion of the flexible conductor.

10. The magnetic stimulator apparatus according to claim 25 wherein the flexible conductor further defines a neck portion.

11. The magnetic stimulator apparatus according to claim 10, wherein the neck portion comprises a longitudinal length and the light guide substantially aligns with at least a portion of the neck portion.

12. The magnetic stimulator apparatus according to claim 25, wherein the light guide is an elongate flexible element.

13. The magnetic stimulator apparatus according to claim 9, wherein the light guide aligns with at least a portion of a turn of the coil.

14. The magnetic stimulator apparatus according to claim 25, wherein the light guide is at least partially secured to the flexible conductor.

15. The magnetic stimulator apparatus according to claim 25 further comprising a housing to house the flexible conductor and light guide.

16. The magnetic stimulator apparatus according to claim 25, wherein the light guide is a light pipe.

17. The magnetic stimulator apparatus according to claim 25 further comprising a charging circuit, a capacitor and a discharge control for allowing discharge of the capacitor through the flexible conductor.

18. The magnetic stimulator apparatus according to claim 25, wherein the flexible conductor further comprises a second multiple turn coil.

19. The A magnetic stimulator apparatus according to claim 18, wherein the second coil is in a side-by-side configuration with the first coil.

20. The magnetic stimulator apparatus according to claim 18, wherein the light guide is in communication with the flexible conductor forming the second multiple turn coil.

21. The magnetic stimulator apparatus according claim 18, wherein the conductor comprises a bridge extending between the first and second coils, and the light guide is in communication with the bridge.

22. The magnetic stimulator apparatus according to claim 21, wherein the light guide is substantially aligned with at least a portion of the bridge.

23. A method of identifying at least partial failure of a flexible conductor in a magnetic stimulator arrangement for the magnetic stimulation of neuromuscular tissue using the magnetic stimulator apparatus according to claim 24, the method comprising monitoring for failure of the light guide.

24. The magnetic stimulator apparatus according to claim 1, wherein the light guide is configured to preferentially fail in the event of at least partial failure of the conductor.

25. A magnetic stimulator apparatus for the magnetic stimulation of neuromuscular tissue comprising:
a flexible conductor arranged to define a multiple turn coil; and
a light guide;
wherein the light guide is configured to preferentially fail in the event of at least partial failure of the conductor; and
wherein the light guide is contiguous with at least a portion of the flexible conductor.
